(19) United States
(12) Patent Application Publication
(10) Pub. No.: US 2011/0092582 A1
(43) Pub. Date: Apr. 21, 2011

(54) New Terpenes And Macrocycles

(75) Inventors: Delphine Jacquot, Braunschweig (DE); Oliver Poeschke, Wiesbaden (DE); Christa Burger, Darmstadt (DE)

(73) Assignee: MERCK PATENT GESELLSCHAFT

(21) Appl. No.: 12/996,408

(22) PCT Filed: May 8, 2009
(86) PCT No.: PCT/EP09/03286

§ 371 Date: Dec. 6, 2010

(30) Foreign Application Priority Data
Jun. 7, 2008 (EP) 08010413.6

(51) Int. Cl. A61K 31/395; A61K 31/215; A61K 31/16; A61K 31/122; C07D 267/00; C07C 69/74; C07C 233/77; C07C 233/64; C07C 49/788

(52) U.S. Cl. 514/450; 514/529; 514/623; 514/691; 540/454; 560/117; 564/188; 568/338; 568/373

(57) ABSTRACT

Novel terpenes and macrocycles are activators of TGR5 and can be used for the prevention and/or treatment of Diabetes Type 2, obesity, neuropathy and/or nephropathy.

### FIELD OF INVENTION

This invention relates to novel natural products and related semisynthetic derivatives, pharmaceutical compositions containing such compounds and the use of those compounds and/or compositions for treating diseases and conditions in man and other mammals mediated by TGR5 signaling, either alone or in combination with other anti-diabetic treatments.

The invention had the object of finding novel compounds having valuable properties, in particular those which can be used for the preparation of medicaments.

The present invention relates to compounds that are useful in the treatment and/or prevention of diseases mediated by deficient levels of GLP-1, such as diabetes mellitus, and methods of preparing such compounds. Also provided are methods of treating diseases and disorders which can be treated by activating TGR5, comprising administering an effective amount of a compound of this invention.

The identification of small compounds which specifically activate, regulate and/or modulate signal transduction of TGR5 is therefore desirable and an aim of the present invention. Moreover, aim of this invention was the preparation of new compounds for the prevention and/or treatment of Diabetes Type 2, obesity, neuropathy and/or nephropathy.

Surprisingly we have found that new terpenes and macrocycles isolated from natural sources as well as semi-synthetic derivatives activate TGR5; therefore, these compounds are especially suitable for the prevention and treatment of Diabetes Type 2, obesity, neuropathy and/or nephropathy. It has been found that the compounds according to the invention and salts thereof have very valuable pharmacological properties while being well tolerated.

The present invention therefore relates to compounds according to the invention as medicaments and/or medicament active ingredients in the treatment and/or prophylaxis of the said diseases and to the use of compounds according to the invention for the preparation of a pharmaceutical for the treatment and/or prophylaxis of the said diseases and also to a process for the treatment of the said diseases which comprises the administration of one or more compounds according to the invention to a patient in need of such an administration.

The host or patient may belong to any mammal species, for example a primate species, particularly humans; rodents, including mice, rats and hamsters; rabbits; horses, cows, dogs, cats, etc. Animal models are of interest for experimental investigations, where they provide a model for the treatment of a human disease.

### BACKGROUND OF THE INVENTION

Type 2 diabetes is characterized by a defective insulin release in response to glucose, a defective response to insulin by liver, fat and muscle cells, enhanced glucose blood levels, reduced glucose tolerance, enhanced insulin blood levels, enhanced triglycerides and enhanced fat tissue. Inflammatory cytokines are enhanced in Diabetes patients. Overweight is one of main key predictors for Diabetes type 2 and a good correlation between obesity and abnormal glucose tolerance is described in several studies. A defective insulin release in response to glucose is a major defect in type 2 diabetes. This is the consequence of an altered glucose metabolism in the beta cells, but also to a decreased release of potentiating factors, such as GLP-1. GLP-1 is an incretin hormone, released by the gut in response to meals. Its main activity is to amplify the insulin response to glucose but most of its other actions could contribute to improve glycemic control in type 2 diabetic patients: Stimulation of islet cell proliferation and neogenesis, inhibition of beta cell apoptosis, inhibition of glucagon secretion, delay in gastric emptying, inhibition of food and water intake, increase in glucose uptake in peripheral tissues. GLP-1 was recently identified as a neuroprotective/neurotrophic factor and involved in the improvement of learning and memory.

Numerous studies have shown the efficacy of treatment with GLP-1 on glycemia, glucose-induced insulin secretion, beta cell growth and gastric emptying in animal models of type 2 diabetes. In type 2 diabetic patients, sc or iv GLP-1 injection (3 times a day) or long-term infusion strongly improved glycemic control. This improvement of glycemic control is also observed when GLP-1 is co-administered with a sulfonylurea, compared to the treatment with the sulfonyurea alone.

Reduction of overweight is a classical successful treatment for Diabetes if achievable. Feeding of bile acids to rodents to reduce body weight as well as to improve glucose and triglyceride levels was described some time ago (Watanabe et al. 2006, Nature 439: 484-489). Triglyceride accumulation in liver and muscles was prevented by CA feeding and glucose uptake in muscles was enhanced. The T3 and T4 levels were not substantially affected by the diet (Watanabe et al. 2006, Nature 439: 484-489).

Recently, TGR5 (Strausberg et al. 2002, PNAS 99: 16899-16903; Takeda et al. 2002, FEBS Lett. 520:97-101), a Gs-protein-coupled receptor (GenBank Accession No. Q8TDU6; NM_{—}170699) first found by searches in the human genome databases for orphan receptor in 2002, was found to have a function in Diabetes. Katsuma et al. showed that TGR5 expressing enteroendocrine cells secreted GLP-1 after stimulation with bile acids (4 to 5 times more) (Katsuma et al. 2005, BBRC 329: 386-390). GLP-1 secretion was reduced by TGR5 siRNA and enhanced by TGR5 overexpression.

TGR5 cDNA from human, mouse and rat showed 82-91% homology. In human, TGR5 was found to be expressed in placenta, leukocytes, spleen, kidney, heart and muscle as well as in the whole digestive system (Maruyama et al. 2002, BBRC 298: 714-719). The gene of TGR5 contains no introns and encodes for a 330 AA protein (Strausberg et al. 2002, PNAS 99: 16899-16903; Takeda et al. 2002, FEBS Lett. 520:97-101; Maruyama et al. 2002, BBRC 298: 714-719). Maruyama et al. described in 2002 that TGR5 overexpressed in HEK293 cells as well as endogenously expressed in enteroendocrine cell lines responded to several bile acids with cAMP production. They named TGR5BG37 (Maruyama et al. 2002, BBRC 298: 714-719). The most potent ligand were lithocholic acid (LCA) with EC50 of 35 nM followed by deoxycholic acid (DCA) with EC50 of 575 nM. Fasting bile acid levels in serum are below 5 μM, whereas postprandial levels increase up to 15 μM. Thus, bile acids look like the endogenous ligands for TGR5. In 2003, Kawamata et al. published TGR5 sequences of human, bovine, rabbit, rat and mouse with an expression pattern as described. In addition they showed a high expression in CD14+ resting monocytes (Kawamata et al. 2002, JBC 278(11): 9435-9440). In rabbit alveolar macrophages as well as TGR5 overexpressing THP-1 cells, treatment with bile acids suppressed LPS induced cytokine production (Kawamata et al. 2002, JBC 278(11): 9435-9440). Phagocytosis of macrophages was suppressed as well. LCA and taurine conjugated LCA (TLCA) were the most potent ligands with EC50 of 10 μM. MAP Kinase was activated by bile acids. Bile acid concentrations exceed 100 μM by viral hepatitis and biliary cirrhosis.

In January 2006 Watanabe et al showed that TGR5 is expressed in the murine and human most thermogenically important tissues, BAT and skeletal muscle together with D2 (type 2 iodothyronine deioddinase), the enzyme reasonable for conversion of T4 to T3 Thyroid hormone. Feeding of cholic acid (CA) in mice leads to a reversal of weight gain by enhanced energy expenditure, probably via TGR5. Under CA feeding, TGR5 is overexpressed and the activity of D2 is enhanced via TGR5 (Watanabe et al. 2006, Nature 439: 484-489). In BAT and human skeletal muscle cells cAMP level is induced by bile acids and by an agonist for TGR5. Glucose as well as insulin tolerance was improved under CA treatment.

TGR5 is therefore a target which has several positive effects on Diabetes. It stimulates the secretion of GLP-1, reduces the amount of inflammatory cytokines and via indirect effects it enhances the energy expenditure of muscle cells.

### SUMMARY OF THE INVENTION

The invention relates to compounds selected from the group

a) compounds of formula I

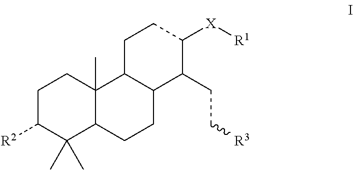

in which
R¹ denotes OH, OA, OAr, OC(O)A, OC(O)Ar, NH₂, NHA, NA₂, NHAr, NHC(O)A or NHC(O)Ar,
R² denotes O, OH, OA, OAr, OC(O)A, OC(O)Ar, NH₂, NHA, NA₂, NHAr, NHC(O)A or NHC(O)Ar,
R³ denotes H, A or Ar,
X denotes CH₂ or CO,
R⁴ denotes H or alkyl having 1-4 C atoms,
[symbol] denotes a single or double bond,
A denotes unbranched or branched alkyl having 1-10 C atoms, in which 1-7 H atoms may be replaced by F and/or Cl,
Ar denotes phenyl, naphthyl or biphenyl, each of which is unsubstituted or mono-, di- or trisubstituted by Hal, A, OR⁴,
N(R⁴)₂, NO₂, CN, COOR⁴, CON(R⁴)₂, NR⁴COA, NR⁴SO₂A, COR⁴, SO₂N(R⁴)₂, S(O)ₙA, [C(R⁴)₂]ₘCOOR⁴ and/or O[C(R⁴)₂]ₘCOOR⁴,
m denotes 0, 1, 2, 3 or 4,
n denotes 0, 1 or 2,
Hal denotes F, Cl, Br or I;
   b) compounds of formula II

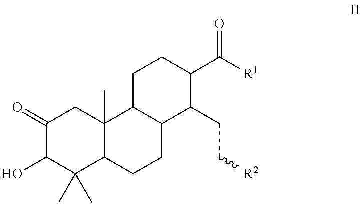

in which.
R¹ denotes OH, OA, OAr, NH₂, NHA, NA₂, NHAr, NH(CH₂)ₚNH₂, NH(CH₂)ₚNHA, NH(CH₂)ₚNA₂,
R² denotes H, A or Ar,
R⁴ denotes H or alkyl having 1-4 C atoms,
[symbol] denotes a single or double bond,
A denotes unbranched or branched alkyl having 1-10 C atoms, in which 1-7 H atoms may be replaced by F and/or Cl,
Ar denotes phenyl, naphthyl or biphenyl, each of which is unsubstituted or mono-, di- or trisubstituted by Hal, A, OR⁴, N(R⁴)₂, NO₂, CN, COOR⁴, CON(R⁴)₂, NR⁴COA, NR⁴SO₂A, COR⁴, SO₂N(R⁴)₂, S(O)ₙA, [C(R⁴)₂]ₘCOOR⁴ and/or O[C(R⁴)₂]ₘCOOR⁴,
m denotes 0, 1, 2, 3 or 4,
n denotes 0, 1 or 2,
p denotes 1, 2, 3 or 4,
Hal denotes F, Cl, Br or I;
   c) compounds of formula III

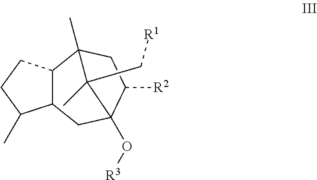

in which
R¹ denotes O, OH, OA, OAr, OC(O)A, OC(O)Ar, NH₂, NHA, NA₂, NHAr, NHC(O)A or NHC(O)Ar,
R² denotes O, OH, OA, OAr, OC(O)A, OC(O)Ar, NH₂, NHA, NA₂, NHAr, NHC(O)A or NHC(O)Ar,
R³ denotes H, A or Ar,
R⁴ denotes H or alkyl having 1-4 C atoms,
[symbol] denotes a single or double bond,
A denotes unbranched or branched alkyl having 1-10 C atoms, in which 1-7 H atoms may be replaced by F and/or Cl,
Ar denotes phenyl, naphthyl or biphenyl, each of which is unsubstituted or mono-, di- or trisubstituted by Hal, A, OR⁴, N(R⁴)₂, NO₂, CN, COOR⁴, CON(R⁴)₂, NR⁴COA, NR⁴SO₂A, COR⁴, SO₂N(R⁴)₂, S(O)ₙA, [C(R⁴)₂]ₘCOOR⁴ and/or O[C(R⁴)₂]ₘCOOR⁴,
m denotes 0, 1, 2, 3 or 4,
n denotes 0, 1 or 2,
Hal denotes F, Cl, Br or I;
   d) compounds of formula IV

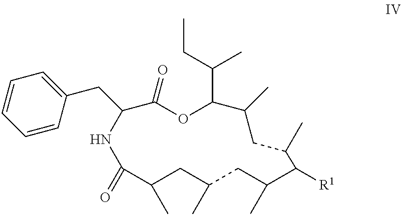

in which
R¹ denotes OH, OA, OAr, OC(O)A, OC(O)Ar, NH₂, NHA, NA₂, NHAr, NHC(O)A or NHC(O)Ar,
R⁴ denotes H or alkyl having 1-4 C atoms,
[symbol] denotes a single or double bond,
A denotes unbranched or branched alkyl having 1-10 C atoms, in which 1-7 H atoms may be replaced by F and/or Cl,
Ar denotes phenyl, naphthyl or biphenyl, each of which is unsubstituted or mono-, di- or trisubstituted by Hal, A, OR⁴, N(R⁴)₂, NO₂, CN, COOR⁴, CON(R⁴)₂, NR⁴COA, NR⁴SO₂A, COR⁴, SO₂N(R⁴)₂, S(O)ₙA, [C(R⁴)₂]ₘCOOR⁴ and/or O[C(R⁴)₂]ₘCOOR⁴,
m denotes 0, 1, 2, 3 or 4,
n denotes 0, 1 or 2,
Hal denotes F, Cl, Br or I;
   and pharmaceutically usable salts and stereoisomers thereof, including mixtures thereof in all ratios.

The invention relates to the compounds of the formula I, II, III and IV and salts thereof and to a process for the preparation of compounds of the formula I, II and IV and pharmaceutically usable salts and stereoisomers thereof,

characterised in that a radical R¹ is converted into another radical R¹ by converting a hydroxyl or alkoxy group to an optionally substituted amino group
and/or
a base or acid of the formula I, II or IV is converted into one of its salts.

The invention furthermore relates to a process for the preparation of compounds of the formula III and pharmaceutically usable salts and stereoisomers thereof,

characterised in that
a carbonyl group is converted by reductive amination into an optionally substituted amino group
and/or
a base or acid of the formula III is converted into one of its salts.

Compounds of formula I, II, III and IV also mean the pharmaceutically usable derivatives and solvates.

The invention also relates to the stereoisomers (E, Z isomers) and the hydrates and solvates of these compounds. Solvates of the compounds are taken to mean adductions of inert solvent molecules onto the compounds which form owing to their mutual attractive force. Solvates are, for example, mono- or dihydrates or alcoholates.

Pharmaceutically usable derivatives is taken to mean, for example, the salts of the compounds according to the invention and also so-called prodrug compounds.

Prodrug derivatives is taken to mean compounds of the formula I, II, III, IV which have been modified, with, for example, alkyl or acyl groups, sugars or oligopeptides and which are rapidly cleaved in the organism to form the active compounds according to the invention.

These also include biodegradable polymer derivatives of the compounds according to the invention, as is described, for example, in Int. J. Pharm. 115, 61-67 (1995).

The expression “effective amount” means the amount of a medicament or pharmaceutical active ingredient which causes a biological or medical response which is sought or aimed at, for example by a researcher or physician, in a tissue, system, animal or human.

In addition, the expression “therapeutically effective amount” means an amount which, compared with a corresponding subject who has not received this amount, has the following consequence:

improved treatment, healing, prevention or elimination of a disease, syndrome, condition, complaint, disorder or prevention of side effects or also the reduction in the progress of a disease, condition, disorder or side effects or also the reduction in the progress of a disease, condition or disorder.

The expression “therapeutically effective amount” also encompasses the amounts which are effective for increasing normal physiological function.

The invention also relates to mixtures of the compounds of the formula I, II, Ill, IV according to the invention, for example mixtures of two diastereomers, for example in the ratio 1:1, 1:2, 1:3, 1:4, 1:5, 1:10, 1:100 or 1:1000.

These are particularly preferably mixtures of stereoisomeric compounds.

For all radicals which occur more than once, their meanings are independent of one another.

Above and below, the radicals and parameters R¹, R², R³, R⁴ and . . . (single or double bond) have the meanings indicated for the formula I, II III, IV unless expressly indicated otherwise.

If R¹ and/or R² means “O”, i.e. ═O (carbonyl group).

A denotes alkyl, is unbranched (linear) or branched, and has 1, 2, 3, 4, 5, 6, 7, 8, 9 or 10 C atoms. A preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, further preferably, for example, trifluoromethyl.

A very particularly preferably denotes alkyl having 1, 2, 3, 4, 5 or 6 C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl.

Ar denotes, for example, phenyl, o-, m- or p-tolyl, o-, m- or p-ethylphenyl, o-, m- or p-propylphenyl, o-, m- or p-isopropylphenyl, o-, m- or p-tert.-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-acetamidophenyl, o-, m- or p-methoxyphenyl, o-, m- or p-ethoxyphenyl, o-, m- or p-ethoxycarbonylphenyl, o-, m- or p-(N,N-dimethylamino)phenyl, o-, m- or p-(N,N-dimethylaminocarbonyl)-phenyl, o-, m- or p-(N-ethylamino)phenyl, o-, m- or p-(N,N-diethylamino)-phenyl, o-, m- or p-fluorophenyl, o-, m- or p-bromophenyl, o-, m- or p-chlorophenyl, o-, m- or p-(methylsulfonamido)phenyl, o-, m- or p-(methylsulfonyl)phenyl, o-, m- or p-cyanophenyl, o-, m- or p-ureidophenyl, o-, m- or p-formylphenyl, o-, m- or p-acetylphenyl, o-, m- or p-aminosulfonylphenyl, o-, m- or p-carboxyphenyl, o-, m- or p-carboxymethylphenyl, o-, m- or p-carboxymethoxyphenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl.

The compounds of the formula I, II, III, IV may have one or more chiral centres and can therefore occur in various stereoisomeric forms. The formula I encompasses all these forms.

Accordingly, the invention relates, in particular, to the compounds of the formula I, II, III, IV in which at least one of the said radicals has one of the preferred meanings indicated above. Some preferred groups of compounds may be expressed by the following sub-formulae Ia, IIa, IIIa and IVa, which conform to the formula I, II, III and IV and in which the radicals not designated in greater detail have the meaning indicated for the formula I, II, III and IV but in which

### in Ia

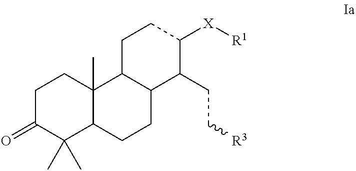 [0081] in which
R¹ denotes OH, OA, NH₂, NHA or NA₂,
R³ denotes H or A,
X denotes CH₂ or CO,
[symbol] denotes a single or double bond,
A denotes unbranched or branched alkyl having 1-10 C atoms, in which 1-7 H atoms may be replaced by F and/or Cl;

### in IIa

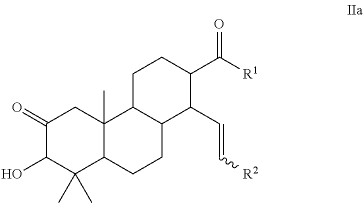 [0088] in which
R¹ denotes OH, OA, NH₂, NHA, NA₂, NH(CH₂)ₚNHA, NH(CH₂)ₚNA₂,
R² denotes H or A,
[symbol] denotes a single or double bond,
A denotes unbranched or branched alkyl having 1-10 C atoms, in which 1-7 H atoms may be replaced by F and/or Cl,
p denotes 1, 2, 3 or 4;

### in IIIa

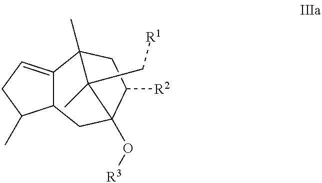 [0095] in which
R¹ denotes O, OH, OA, NH₂, NHA or NA₂,
R² denotes O, OH or OA,
R³ denotes H or A,
[symbol] denotes a single or double bond,
A denotes unbranched or branched alkyl having 1-10 C atoms, in which 1-7 H atoms may be replaced by F and/or Cl;
   in IVa in which
R¹ denotes OH, OA, NH₂, NHA or NA₂,
[symbol] denotes a single or double bond,
A denotes unbranched or branched alkyl having 1-10 C atoms, in which 1-7 H atoms may be replaced by F and/or Cl;
   and pharmaceutically usable, salts and stereoisomers thereof, including mixtures thereof in all ratios.

The compounds according to the invention and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants known per se, which are not mentioned here in greater detail.

If desired, the starting materials can also be formed in situ so that they are not isolated from the reaction mixture, but instead are immediately converted further into the compounds according to the invention.

The starting compounds are generally known. If they are novel, however, they can be prepared by methods known per se.

Compounds of the formula I, II and IV

can preferably be obtained by converting a radical R¹ into another radical R¹ by converting a hydroxyl or alkoxy group to an optionally substituted amino group.

The reaction is carried out by methods which are known to the person skilled in the art.

The reaction is generally carried out in an inert solvent.

Suitable inert solvents are, for example, hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Depending on the conditions used, the reaction time is between a few minutes and 14 days, the reaction temperature is between about −30° and 140°, normally between −10° and 110°, in particular between about 20° and about 100°.

Furthermore, free amino groups can be acylated in a conventional manner using an acid chloride or anhydride or alkylated using an unsubstituted or substituted alkyl halide, advantageously in an inert solvent, such as dichloromethane or THF, and/or in the presence of a base, such as triethylamine or pyridine, at temperatures between −60 and +30°. Ester groups can be saponified, for example, using NaOH or KOH in water, water/THF or water/dioxane at temperatures between 0 and 100′. Carboxylic acids can be converted, for example using thionyl chloride, into the corresponding carboxylic acid chlorides, and the latter can be converted into carboxamides. Elimination of water therefrom in a known manner gives carbonitriles.

### Pharmaceutical Salts and Other Forms

The said compounds according to the invention can be used in their final non-salt form. On the other hand, the present invention also encompasses the use of these compounds in the form of their pharmaceutically acceptable salts, which can be derived from various organic and inorganic acids and bases by procedures known in the art. Pharmaceutically acceptable salt forms of the compounds of the formula I, II, III and IV are for the most part prepared by conventional methods. If the compound of the formula I, II, III and IV contains a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example potassium ethoxide and sodium propoxide; and various organic bases, such as piperidine, diethanolamine and N-methylglutamine. The aluminium salts of the compounds of the formula I, II, III and IV are likewise included. In the case of certain compounds of the formula I, II, III and IV, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as acetate, trifluoroacetate, tartrate, maleate, succinate, citrate, benzoate, salicylate, ascorbate and the like. Accordingly, pharmaceutically acceptable acid-addition salts of the compounds of the formula I, II, III and IV include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate, but this does not represent a restriction.

Furthermore, the base salts of the compounds according to the invention include aluminium, ammonium, calcium, copper, iron(III), iron(II), lithium, magnesium, manganese(III), manganese(II), potassium, sodium and zinc salts, but this is not intended to represent a restriction. Of the above-mentioned salts, preference is given to ammonium; the alkali metal salts sodium and potassium, and the alkaline earth metal salts calcium and magnesium. Salts of the compounds of the formula I, II, III and IV which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris(hydroxymethyl)methylamine (tromethamine), but this is not intended to represent a restriction.

Compounds of the present invention which contain basic nitrogen-containing groups can be quaternised using agents such as (C₁-C₄)alkyl halides, for example methyl, ethyl, isopropyl and tea-butyl chloride, bromide and iodide; di(C₁-C₄)alkyl sulfates, for example dimethyl, diethyl and diamyl sulfate; (C₁₀-C₁₈)alkyl halides, for example decyl, dodecyl, lauryl, myristyl and stearyl chloride, bromide and iodide; and aryl(C₁-C₄)alkyl halides, for example benzyl chloride and phenethyl bromide. Both water- and oil-soluble compounds according to the invention can be prepared using such salts.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine, but this is not intended to represent a restriction.

The acid-addition salts of basic compounds of the formula I, II, III and IV are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.

As mentioned, the pharmaceutically acceptable base-addition salts of the compounds of the formula I, II, III and IV are formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanolamine, ethylenediamine, N-methyl-D-glucamine and procaine.

The base-addition salts of acidic compounds according to the invention are prepared by bringing the free acid form into contact with a sufficient amount of the desired base, causing the formation of the salt in a conventional manner. The free acid can be regenerated by bringing the salt form into contact with an acid and isolating the free acid in a conventional man ner. The free acid forms differ in a certain respect from the corresponding salt forms thereof with respect to certain physical properties, such as solubility in polar solvents; for the purposes of the invention, however, the salts otherwise correspond to the respective free acid forms thereof.

If a compound according to the invention contains more than one group which is capable of forming pharmaceutically acceptable salts of this type, the invention also encompasses multiple salts. Typical multiple salt forms include, for example, bitartrate, diacetate, difumarate, dimeglumine, diphosphate, disodium and trihydrochloride, but this is not intended to represent a restriction.

With regard to that stated above, it can be seen that the expression “pharmaceutically acceptable salt” in the present connection is taken to mean an active ingredient which comprises a compound of the formula I in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

Compounds of the formula I, II, III and IV according to the invention may be chiral owing to their molecular structure and may accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitably N-protected amino acids (for example N-benzoylproline or N-benzenesulfonylproline), or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3.

The invention furthermore relates to the use of the compounds and/or physiologically acceptable salts thereof for the preparation of medicament (pharmaceutical composition), in particular by non-chemical methods. They can be converted into a suitable dosage form here together with at least one solid, liquid and/or semi-liquid excipient or adjuvant and, if desired, in combination with one or more further active ingredients.

The invention furthermore relates to medicaments comprising at least one compound according to the invention and/or pharmaceutically usable salts and stereoisomers thereof, including mixtures thereof in all ratios, and optionally excipients and/or adjuvants.

Pharmaceutical formulations can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinyl-pyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre specified amount of the compounds. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be en capsulated in microcapsules. The formulation can also be prepared in such a way that the release is extended or retarded, such as, for example, by coating or embedding of particulate material in polymers, wax and the like.

The compounds according to the invention and salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds according to the invention and the salts, solvates and physiologically functional derivatives thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formula tion is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary.

Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the present invention depends on a number of factors, including, for example, the age and weight of the human or animal, the precise disease condition which requires treatment, and its severity, the nature of the formulation and the method of administration, and is ultimately determined by the treating doctor or vet. However, an effective amount of a compound according to the invention is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of other conditions mentioned above.

The invention furthermore relates to medicaments comprising at least one compound according to the invention and/or pharmaceutically usable salts and stereoisomers thereof, including mixtures thereof in all ratios, and at least one further medicament active ingredient.

The invention also relates to a set (kit) consisting of separate packs of
(a) an effective amount of a compound according to the invention and/or pharmaceutically usable salts and stereoisomers thereof, including mixtures thereof in all ratios,
   and
(b) an effective amount of a further medicament active ingredient.

The set comprises suitable containers, such as boxes, individual bottles, bags or ampoules. The set may, for example, comprise separate ampoules, each containing an effective amount of a compound according to the invention and/or pharmaceutically usable salts and stereoisomers thereof, including mixtures thereof in all ratios,

and an effective amount of a further medicament active ingredient in dissolved or lyophilised form.

### Use

The present compounds are suitable as pharmaceutical active ingredients for mammals, in particular for humans, in the treatment of Diabetes Type 2, obesity, neuropathy and/or nephropathy.

The invention thus relates to the use of compounds according to claim 1 and to pharmaceutically usable derivatives, solvates and stereoisomers, including mixtures thereof in all ratios, for the preparation of a medicament for the treatment of Diabetes Type 2, obesity, neuropathy and/or nephropathy.

The compounds of the present invention can be used as prophylactics or therapeutic agents for treating diseases or disorders mediated by deficient levels of GLP-1 activity or which can be treated by activating TGR5 including, but not limited to, diabetes mellitus, impaired glucose tolerance, IFG (impaired fasting glucose) and IFG (impaired fasting glycemia), as well as other diseases and disorders such as those discussed below. Furthermore, the compounds of the present invention can be also used to prevent the progression of the borderline type, impaired glucose tolerance, IFG (impaired fasting glucose) or IFG (impaired fasting glycemia) to diabetes mellitus.

The compounds of the present invention can be also used as prophylactics or therapeutic agents of diabetic complications such as, but not limited to, neuropathy, nephropathy, retinopathy, cataract, macroangiopathy, osteopenia, diabetic hyperosmolar coma), infectious diseases (e.g., respiratory infection, urinary tract infection, gastrointestinal tract infection, dermal soft tissue infection, lower limb infection etc.), diabetic gangrene, xerostomia, decreased sense of hearing, cerebrovascular disease, peripheral circulatory disturbance, etc.

The compounds of the present invention can be also used as prophylactics or therapeutic agents in the treatment of diseases and disorders such as, but not limited to, obesity, metabolic syndrome (syndrome X), hyperinsulinemia, hyperinsulinemia-induced sensory disorder, dyslipoproteinemia (abnormal lipoproteins in the blood) including diabetic dyslipidemia, hyperlipidemia, hyperlipoproteinemia (excess of lipoproteins in the blood) including type I, II-a (hypercholesterolemia), II-b, III, IV (hypertriglyceridemia) and V (hypertriglyceridemia), low HDL levels, high LDL levels, atherosclerosis and its sequelae, vascular restenosis, neurodegenerative disease, depression, CNS disorders, liver steatosis, osteoporosis, hypertension, renal diseases (e.g., diabetic nephropathy, glomerular nephritis, glomeruloscierosis, nephrotic syndrome, hypertensive nephrosclerosis, terminal renal disorder etc.), myocardiac infarction, angina pectoris, and cerebrovascular disease (e.g., cerebral infarction, cerebral apoplexy).

The compounds of the present invention can be also used as prophylactics or therapeutic agents in the treatment of diseases and disorders such as, but not limited to, osteoporosis, fatty liver, hypertension, insulin resistant syndrome, inflammatory diseases (e.g., chronic rheumatoid arthritis, spondylitis deformans, osteoarthritis, lumbago, gout, postoperative or traumatic inflammation, remission of swelling, neuralgia, pharyngolaryngitis, cystitis, hepatitis (including non-alcoholic steatohepatitis), pneumonia, inflammatory colitis, ulcerative colitis), pancreatitis, visceral obesity syndrome, cachexia (e.g., carcinomatous eachexia, tuberculous cachexia, diabetic cachexia, hemopathic cachexia, endocrinopathic cachexia, infectious cachexia, cachexia induced by acquired immunodeficiency syndrome), polycystic ovary syndrome, muscular dystrophy, tumor (e.g., leukemia, breast cancer, prostate cancer, skin cancer etc.), irritable bowel syndrome, acute or chronic diarrhea, spondylitis deformans, osteoarthritis, remission of swelling, neuralgia, pharyngolaryngitis, cystitis, SIDS, and the like.

The compounds of the present invention can be used in combination with one or more additional drugs such as described below. The dose of the second drug can be appropriately selected based on a clinically employed dose. The proportion of the compound of formula I, II, III and IV and the second drug can be appropriately determined according to the administration subject, the administration route, the target disease, the clinical condition, the combination, and other factors. In cases where the administration subject is a human, for instance, the second drug may be used in an amount of 0.01 to 100 parts by weight per part by weight of the compound of formula I, II, III and IV.

The second compound of the pharmaceutical combination formulation or dosing regimen preferably has complementary activities to the compound of formula I, II, III and IV such that they do not adversely affect each other. Such drugs are suitably present in combination in amounts that are effective for the purpose intended. Accordingly, another aspect of the present invention provides a composition comprising a compound of formula I, II, III and IV, or a solvate, metabolite, or pharmaceutically acceptable salt or prodrug thereof, in combination with a second drug, such as described herein.

The compound of formula I, II, III and IV and the additional pharmaceutically active agent(s) may be administered together in a unitary pharmaceutical composition or separately and, when administered separately this may occur simultaneously or sequentially in any order. Such sequential administration may be close in time or remote in time The amounts of the compound of formula I, II, III and IV and the second agent(s) and the relative timings of administration will be selected in order to achieve the desired combined therapeutic effect.

The combination therapy may provide “synergy” and prove “synergistic”, i.e., the effect achieved when the active ingredients used together is greater than the sum of the effects that results from using the compounds separately. A synergistic effect may be attained when the active ingredients are (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The compounds of the present invention can be used, for example in combination with additional drug(s) such as a therapeutic agent for diabetes mellitus, and/or a therapeutic agent for diabetic complications, as defined above.

Examples of known therapeutic agents for diabetes mellitus which can be used in combination with a compound of formula I, II, III and IV include insulin preparations (e.g., animal insulin preparations extracted from the bovine or swine pancreas; human insulin preparations synthesized by a genetic engineering technique using Escherichia coli or a yeast), a fragment of insulin or derivatives thereof (e.g., INS-i), agents for improving insulin resistance (e.g., pioglitazone hydrochloride, troglitazone, rosiglitazone or its maleate, GI-262570, JTT-50 1, MCC-555, YM-440, KRP-297, CS-Oil, FK-614), alpha-glucosidase inhibitors (e.g., voglibose, acarbose, miglitol, emiglitate), biguanides (e.g., phenformin, metformin, buformin), insulin secretagogues [sulfonylureas (e.g., tolbutamide, glibenclamide, gliclazide, chiorpropamide, tolazamide, acetohexamide, glyclopyramide, glimepiride, glipizide, glybuzole), repaglinide, nateglinide, mitiglinide or its calcium salt hydrate, GLP-1J, dipeptidylpeptidase IV inhibitors (e.g., NVP-DPP-278, PT-100), beta-3 agonists (e.g., CL-3 16243, SR-58611-A, UL-TG-307, SB-226552, AJ-9677, BMS-196085, AZ-40140 etc.), amylin agonists (e.g., pramlintide), phosphotyrosine phosphatase inhibitors (e.g., vanadic acid), gluconeogenesis inhibitors (e.g., glycogen phosphorylase inhibitors, glucose-6-phosphatase inhibitors, glucagon antagonists), SGLT (sodium-glucose cotransporter) inhibitors (e.g., T-1095), and the like.

Examples of known therapeutic agents for diabetic complications include aldose reductase inhibitors (e.g., tolrestat, epairestat, zenarestat, zopobestat, minairestat, fidarestat (SNK-860), CT-i 12), neurotrophic factors (e.g., NGF, NT-3, BDNF), neurotrophic factor production secretion promoters, PKC inhibitors (e.g., LY-333531), AGE inhibitors (e.g., ALT946, pimagedine, pyratoxathine, N-phenacylthiazolium bromide (ALT766), EXO-226), active oxygen scavengers (e.g., thioctic acid), and cerebral vasodilators (e.g., tiapuride, mexiletine).

The compounds of the present invention can also be used, for example in combination with antihyperlipidemic agents. Epidemiological evidence has firmly established hyperlipidemia as a primary risk factor in causing cardiovascular disease (CVD) due to atherosclerosis. In recent years, emphasis has been placed on lowering plasma cholesterol levels, and low density lipoprotein cholesterol in particular, as an essential step in prevention of CVD.

Cardiovascular disease is especially prevalent among diabetic subjects, at least in part because of the existence of multiple independent risk factors in this population. Successful treatment of hyperlipidemia in the general population, and in diabetic subjects in particular, is therefore of exceptional medical importance. Examples of antihyperlipidemic agents include statin compounds which are cholesterol synthesis inhibitors (e.g., cerivastatin, pravastatin, simvastatin, lovastatin, atorvastatin, fluvastatin, itavastatin or their salts, etc.), squalene synthase inhibitors or fibrate compounds (e.g., bezafibrate, clofibrate, simfibrate, clinofibrate) having a triglyceride lowering action and the like.

The compounds of the present invention can also be used, for example in combination with hypotensive agents. Hypertension has been associated with elevated blood insulin levels, a condition known as hyperinsulinemia. Insulin, a peptide hormone whose primary actions are to promote glucose utilization, protein synthesis and the formation and storage of neutral lipids, also acts to promote vascular cell growth and increase renal sodium retention, among other things. These latter functions can be accomplished without affecting glucose levels and are known causes of hypertension. Peripheral vasculature growth, for example, can cause constriction of peripheral capillaries, while sodium retention increases blood volume. Thus, the lowering of insulin levels in hyperinsulinemics can prevent abnormal vascular growth and renal sodium retention caused by high insulin levels and thereby alleviates hypertension. Examples of hypotensive agents include angiotensin converting enzyme, inhibitors (e.g., captopril, enalapril, delapril), angiotensin II antagonists (e.g., candesartan cilexetil, losartan, eprosartan, valsantan, termisartan, irbesartan, tasosartan), calcium antagonists (e.g., manidipine, nifedipine, nicardipine, amlodipine, efonidipine), and clonidine.

The compounds of the present invention can be used in combination with antiobesity agents. The term “obesity” implies an excess of adipose tissue. Obesity is a well-known risk factor for the development of many very common diseases such as diabetes, atherosclerosis, and hypertension. To some extent appetite is controlled by discrete areas in the hypothalamus: a feeding centre in the ventrolateral nucleus of the hypothalamus (VLH) and a satiety centre in the ventromedial hypothalamus (VMH). The cerebral cortex receives positive signals from the feeding center that stimulate eating, and the satiety center modulates this process by sending inhibitory impulses to the feeding center. Several regulatory processes may influence these hypothalamic centers. The satiety center may be activated by the increases in plasma glucose and/or insulin that follow a meal. Examples of antiobesity agents include antiobesity drugs acting on the central nervous system (e.g., dexfenfluramine, fenfluramine, phentermine, sibutramine, anfepramon, dexamphetamine, mazindol, phenylpropanolamine, clobenzorex), pancreatic lipase inhibitors (e.g. orlistat), beta-3 agonists (e.g., CL-3 16243, SR-5861 1-A, UL-TG-307, SB-226552, AJ-9677, BMS-196085, AZ-40140), anorectic peptides (e.g., leptin, CNTF (Ciliary Neurotrophic Factor) and cholecystokinin agonists (e.g. lintitript, FPL-1 5849).

Above and below, all temperatures are indicated in ° C. In the following examples, “conventional work-up” means: if necessary, water is added, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rf values on silica gel; eluent: ethyl acetate/methanol 9:1.
Mass spectrometry (MS): EI (electron impact ionisation) M⁺
   FAB (fast atom bombardment) (M+H)⁺
   ESI (electrospray ionisation) (M+H)⁺ (unless indicated otherwise)

### Methods and Materials

### Method 1: Preparative RP-MPEG Method

| MPLC | Kronlab GmbH | | | | |
|---|---|---|---|---|---|
| System | | | | | |
| Data | Prepcon 4.47 | | | | |
| System | | | | | |
| Stationary | Polygoprep 60-50 RP-18 (Macherey & Nagel) | | | | |
| Phase | | | | | |
| Mobile | A: deionized Water | | | | |
| Phase | B: Methanol (p.a.) | | | | |
| | C: Isopropanol | | | | |
| Gradient | time [min] | Flow Rate [ml/min] | % A | % B | % C |
| | 0.0 | 100 | 100 | 0 | 0 |
| | 5.0 | 100 | 100 | 0 | 0 |
| | 5.1 | 130 | 100 | 0 | 0 |
| | 10.0 | 130 | 100 | 0 | 0 |
| | 10.1 | 100 | 80 | 20 | 0 |
| | 18.0 | 100 | ¹ | ² | 0 |
| | 51.0 | 100 | 10 | 90 | 0 |
| | 61.0 | 100 | 10 | 90 | 0 |
| | 61.1 | 150 | 0 | 100 | 0 |
| | 66.0 | 150 | 0 | 100 | 0 |
| | 66.1 | 30 | 0 | 0 | 100 |
| | 70.0 | 30 | 0 | 0 | 100 |
| | 70.1 | 75 | 0 | 0 | 100 |
| | 74.0 | 75 | 0 | 0 | 100 |

| | ¹% A( ) | ²(% B) |
|---|---|---|
| “A3” | 43 | 57 |
| “A1” and “A2” | 36 | 64 |
| “A4” | 37 | 63 |

### Method 2: Analytical HPLC/ELSD/UV Method

| HPLC System | Merck Hitachi | | |
|---|---|---|---|
| Data System | HPLC-Manager D-7000 HSM | | |
| Stationary | Merck Superspher60 RP Select B 125 × 4 mm, 4 μm | | |
| Phase | | | |
| Flow Rate | 1 ml/min | | |
| Detection | ELSD (Sedex 75), UV (Merck, 254 nm) | | |
| Injection | 30 μl | | |
| Volume | | | |
| Mobile Phase: | A: 5 mM ammoniumformiate and 0.1% formic acid | | |
| | B: acetonitrile/methanol = 1:1, 5 mM ammoniumformiate | | |
| | and 0.1% formic acid (pH 3) | | |
| Gradient | time [min] | % A | % B |
| | 00.0 | 85 | 15 |
| | 15.0 | 0 | 100 |
| | 18.0 | 0 | 100 |
| | 18.5 | 85 | 15 |
| | 21.5 | 85 | 15 |

### Method 3: Preparative HPLC Method for EMD 1176097 and EMD 1176099

| HPLC System | SEBOXLight ® | |
|---|---|---|
| Data System | SEPBOX control 2.1.1. | |
| Stationary | KromasilC18, 250 × 50 mm, 10 μm | |
| Phase | | |
| Flow Rate | 109 ml/min | |
| Detection | ELSD (Sedex 75; Gain 5) | |
| | UV (Merck, 250 nm) | |
| Fractionation | Parameter | Remarks |
| | Mode | Time Based Fractionation |
| | Time | 30 sec./Fraction |
| | Window | 7.7-57.7 min. |
| | Number | 100 Fractions |

| Mobile Phase: | A: 0.1% formic acid | | |
|---|---|---|---|
| | B: acetonitrile, 0.1% formic acid (pH 3) | | |
| Gradient | time [min] | % A | % B |
| | 0.00 | 49 | 51 |
| | 57.7 | 30 | 70 |
| | 57.8 | 0 | 100 |
| | 65.8 | 0 | 100 |

### Method 4: Preparative HPLC Method for EMD 1176088

| HPLC System | SEBOXLight ® | |
|---|---|---|
| Data System | SEPBOX control 2.1.1. | |
| Stationary | KromasilC18, 250 × 50 mm, 10 μm | |
| Phase | | |
| Flow Rate | 109 ml/min | |
| Detection | ELSD (Sedex 75; Gain 5) | |
| | UV (Merck, 250 nm) | |
| Fractionation | Parameter | Remarks |
| | Mode | Time Based Fractionation |
| | Time | 30 sec./Fraction |
| | Window | 7.7-57.7 min. |
| | Number | 100 Fractions |

| Mobile Phase: | A: 0.1% formic acid | | |
|---|---|---|---|
| | B: acetonitrile, 0.1% formic acid (pH 3) | | |
| Gradient | time [min] | % A | % B |
| | 0.00 | 62 | 38 |
| | 57.7 | 45 | 55 |
| | 57.8 | 0 | 100 |
| | 65.8 | 0 | 100 |

### Method 5: Preparative HPLC Method for EMD 1.205333

| HPLC System | SEBOXLight ® | |
|---|---|---|
| Data System | SEPBOX control 2.1.1. | |
| Stationary | MerckSelectB, 250 × 50 mm,10 μm | |
| Phase | | |
| Flow Rate | 80 ml/min | |
| Detection | ELSD (Sedex 75; Gain 5) | |
| | UV (Merck, 250 nm) | |
| Fractionation | Parameter | Remarks |
| | Mode | Time Based Fractionation |
| | Time | 30 sec./Fraction |
| | Window | 7.7-57.7 min. |
| | Number | 100 Fractions |

| Mobile Phase: | A: 5 mM ammoniumformiate and 0.1% formic acid | | |
|---|---|---|---|
| | B: acetonitrile/methanol = 1:1, 5 mM ammoniumformiate | | |
| | and 0.1% formic acid (pH 3) | | |
| Gradient for | | | |
| M-9021-C | time [min] | % A | % B |
| | 0.00 | 33 | 67 |
| | 57.7 | 19 | 81 |
| | 57.8 | 0 | 100 |
| | 65.8 | 0 | 100 |

### Method 6: Analytical HPLC/ELSD Method (Purity Determination of Compound)

| HPLC | Merck Hitachi | | |
|---|---|---|---|
| System | | | |
| Data | HPLC-Manager D-7000 HSM | | |
| System | | | |
| Stationary | Merck Supersphere 125 × 4 mm, 5 □m | | |
| Phase | | | |
| Flow Rate | 2 ml/min | | |
| Detection | ELSD (Sedex 75) | | |
| Injection | 30 μl | | |
| Volumen | | | |
| Mobile | A: 5 mM ammoniumformiate and 0.1% formic acid | | |
| Phase: | B: acetonitrile/methanol = 1:1, 5 mM ammoniumformiate and | | |
| | 0.1% formic acid (pH 3) | | |
| Gradient | Time [min] | % A | % B |
| | 00.0 | 85 | 15 |
| | 15.0 | 0 | 100 |
| | 20.0 | 0 | 100 |

| Instrument | MS: Applied Biosystems API 150 | |
|---|---|---|
| | HPLC: Perkin Elmer PE200 | |
| | ELSD: Sedex 75 | |
| Column | Phenomenex Luna C8 (2), 50 × 3 mm, 3 μm | |
| Flow rate | 1.2 ml/min. | |
| Mobile phase | Solvent A: 5 mM ammoniumformiate and 0.1% formic | |
| | acid | |
| | Solvent B: acetonitrile/methanol = 1:1 with 5 mM | |
| | ammoniuniformiate and 0.1% formic acid | |
| | Time [min] | % B |
| | 0.0 | 5 |
| | 6.0 | 100 |
| | 8.0 | 100 |

| | |
|---|---|
| HPLC System | PE series 200 |
| MS System | Applied Biosystems API 150, 165 or 365 |
| Data System | Analyst 1.3 or Masschrom 1.2.1. |
| Stationary | Phenomenex Luna C8, 5 μm, 50 × 4.6 mm |
| Phase | |
| Flow Rate | 1.5 ml/min |
| Detection | (+/(−)-ESI, Fast-Switching-Mode , ELSD (Sedex 75) |
| Injection | 30 μl |
| Volumen | |
| Mobile Phase: | A: 5 mM ammoniumformiate and 0.1% formic acid |
| | B: acetonitrile/methanol = 1:1, 5 mM ammoniumformiate |
| | and 0.1% formic acid (pH 3) |
| Gradient | Adapted to the corresponding polarity window |

| | |
|---|---|
| Instrument | Bruker 400 MHz or 500 MHz |
| Software | XWINNMR 3.1 |
| Solvent | CD₃OD (referenced by lock signal of the solvent |
| Tubes | 5 mm |

### EXAMPLE 1

### 17-hydroxy-12,15-cleistanthadiene-3-one (“A1”)

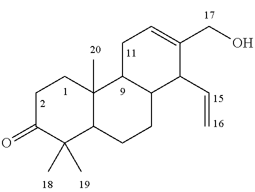

518.5 g of grinded dried plant material (from roots of Tiliaceae Berrya mollis) was extracted with a mixture of methanol and MTBE (1:1, v/v, 3500 MI), filtration with vacuum (Büchner funnel) followed by extraction with methanol (3500 ml) and filtration with vacuum (Büchner funnel). Both extracts were combined (total volume 6650 ml) and covered on the double amount of Celite by evaporation with rotary evaporator (decreasing vacuum: 250 to 50 mbar), leading to 38.1 g of crude extract.

The crude extract was fractionated by RP-MPLC according to Method 1. Fraction D (analysed with Method 2) with retention time window from 33 to 40.5 min was evaporated to yield 0.5 g of pre-purified extract. Fraction D was further purified by RP-HPLC according to Method 3.

Fractions 38 and 39 yielded 8.3 mg of “A1” as an amorphous solid in 99.8% purity (Method 6), Rt (Method 6)=14.31 min;

ESIMS (+): m/z=303 [M+H], 285 [M−H₂O+H];

¹H-NMR (CD₃OD, 400 MHz) and HH—COSY, HMBC, HMQC (CD₃OD, 400 MHz):

| Position | ¹³C [ppm] | ¹H [ppm] | | J [Hz] |
|---|---|---|---|---|
| 1 | 38.0 | 1.44 | m | |
| | | 2.02 | m | |
| 2 | 34.4 | 2.71 | ddd | 14; 13; 6 |
| | | 2.28 | ddd | 14; 6; 3 |
| 3 | 217.8 | — | | |
| 4 | 48.0 | — | | |
| 5 | 55.6 | 1.41 | dd | 14; 2 |
| 6 | 22.3 | 1.49 | m | |
| | | 1.57 | m | |
| 7 | 33.1 | 0.95 | dddd | 12; 12; |
| | | 2.15 | br d | 12; 4 |
| | | | | 12 |
| 8 | 37.4 | 1.31 | m | |
| 9 | 49.2 | 1.10 | m | |
| 10 | 37.1 | — | | |
| 11 | 23.78 | 1.90 | m | |
| | | 2.09 | m | |
| 12 | 122.7 | 5.80 | brs | |
| 13 | 136.6 | — | | |
| 14 | 51.0 | 2.41 | brt | 9 |
| 15 | 140.9 | 5.42 | ddd | 16; 9; 9 |
| 16 | 116.4 | 5.10 | brd | 16 |
| | | 5.08 | brd | 9 |
| 17 | 64.0 | 3.91 | brd | 13 |
| | | 3.82 | brd | 13 |
| 18 | 25.0 | 1.04 | s | |
| 19 | 21.7 | 1.07 | s | |
| 20 | 13.1 | 1.05 | s | |

### EXAMPLE 2

### 15-ene-3-hydroxy-2-oxo-cleistanthan-17-oic acid methylester (“A2”)

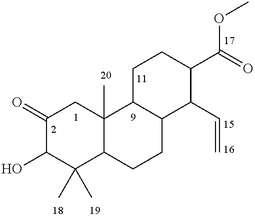

518.5 g of grinded dried plant material (from roots of Tiliaceae Berrya mollis) was extracted with a mixture of methanol and MTBE (1:1, v/v, 3500 ml), filtration with vacuum (Büchner funnel) followed by extraction with methanol (3500 ml) and filtration with vacuum (Büchner funnel). Both extracts were combined (total volume 6650 ml) and covered on the double amount of Celite by evaporation with rotary evaporator (decreasing vacuum: 250 to 50 mbar), leading to 38.1 g of crude extract.

The crude extract was fractionated by RP-MPLC according to Method 1. Fraction D (analyzed with Method 2) with retention time window from 33 to 40.5 min was evaporated to yield 0.5 g of pre-purified extract. Fraction D was further purified by RP-HPLC according to Method 3.

Fractions 61 and 62 yielded 2.1 mg of “A2” as an amorphous solid in 91.1% purity (Method 6), Rt (Method 6)=14.45 min;

HR-ESI-MS: cal. for C₂₁H₃₃O₄ 348.2300. Found 349.2365 [M+H]⁺;

¹H-NMR (CD₃OD, 400 MHz) and HH—COSY, HMBC, HMQC (CD₃OD, 400 MHz):

| Position | ¹³C [ppm] | ¹H [ppm] | | J [Hz] |
|---|---|---|---|---|
| 1 | 52.4 | 2.35 | d | 12.5 |
| | | 2.28 | d | 12.5 |
| 2 | 211.8 | — | | |
| 3 | 83.6 | 3.99 | s | |
| 4 | 45.9 | — | | |
| 5 | 54.5 | 1.62 | m | |
| 6 | 22.3 | 1.44 | m | |
| | | 1.74 | m | |
| 7 | 33.9 | 0.83 | m | |
| | | 1.94 | m | |
| 8 | 30.2 | 1.29 | m | |
| 9 | 56.0 | 1.06 | m | |
| 10 | 43.9 | — | | |
| 11 | 21.1 | 1.41 | m | |
| | | 1.30 | m | |
| 12 | 29.4 | 2.01 | m | |
| | | 1.63 | m | |
| 13 | 46.7 | 2.64 | m | |
| 14 | 51.8 | 1.84 | m | |
| 15 | 141.8 | 5.76 | ddd | 17.0; |
| | | | | 10.0; 9.5 |
| 16 | 116.2 | 5.01 | dd | 17.0; 2.0 |
| | | 4.98 | dd | 10.0 2.0 |
| 17 | 175.8 | — | | |
| 18 | 28.9 | 1.13 | s | |
| 19 | 16.3 | 0.69 | s | |
| 20 | 15.0 | 0.82 | s | |
| OMe | 51.5 | 3.61 | s | |

### 7-Hydroxy-8-oxo-11(1-10)-abeo-1-patchoulene-12-al (“A3”)

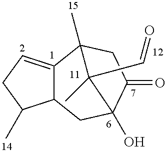

260.5 g of grinded dried plant material (roots of Compositae Cynara humilis) was extracted with a mixture of methanol and MTBE (1:1, v/v, 2200 ml) filtration with vacuum (Büchner funnel) followed by extraction with methanol (2200 ml) and filtration with vacuum (Büchner funnel). Both extracts were combined (total volume 3800 ml) and covered on the double amount of Celite by evaporation with rotary evaporator (decreasing vacuum: 250 to 50 mbar), leading to 34.6 g of crude extract.

The crude extract was fractionated by RP-MPLC according to Method 1. Fraction C (analyzed with Method 2) with retention time window from 25.5 to 33 min was evaporated to yield 11.0 g of pre-purified extract. Fraction C was further purified by RP-HPLC according to Method 4.

Fractions 31 and 32 yielded 19.5 mg of “A3” as an amorphous solid in 77.9% purity (Method 6), Rt (Method 6)=11.51 min;

HR-ESI-MS: cal. for C₁₅H₂₁O₃ 248.1412. Found 249.1498 [M+H]⁺;

¹H-NMR (CD₃OD, 400 MHz) and HH—COSY, HMBC, HMQC (CD₃OD, 400 MHz):

| Position | ¹³C [ppm] | ¹H [ppm] | | J [Hz] |
|---|---|---|---|---|
| 1 | 146.9 | — | | |
| 2 | 121.9 | 5.39 | ddd | 2; 2; 2 |
| 3 | 41.6 | 2.62 | m | 16; 2; 2 |
| | | 1.99 | brd | |
| 4 | 34.2 | 2.45 | brq | 7 |
| 5 | 43.2 | 2.76 | m | |
| 6 | 32.9 | 1.88 | dd | 12.5; 12.5 |
| | | 1.51 | dd | 12.5; 8.5 |
| 7 | 84.5 | — | | |
| 8 | 216.5 | — | | |
| 9 | 47.3 | 2.53 | d | 19 |
| | | 2.22 | d | 19 |
| 10 | 61.6 | — | | |
| 11 | 43.6 | — | | |
| 12 | 207.4 | 9.64 | s | |
| 13 | 9.3 | 1.16 | s | |
| 14 | 17.4 | 0.92 | d | 7 |
| 15 | 17.2 | 1.22 | s | |

### (8E,12E)-3-benzyl-15-sec-butyl-11-hydroxy-6,8,10,12,14-pentamethyl-1-oxa-4-azacyclopentadeca-8,12-diene-2,5-dione (“A4”)

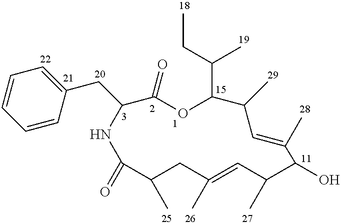

The strain ACD01185fxxx000011 was isolated by a serial dilution method from a soil sample with gras. The strain was grown on malt extract agar and stored with 20% glycerol at −80° C.

The strain ACD01185fxxx000011 was revived on malt extract agar incubating at 25° C. A seed culture of 100 ml MAT2 medium (see Table 1) in a 500 ml Erlenmeyer flask was shaken for 5 days at 30° C. with 160 rpm on an orbital shaker with 5 cm shaking diameter.

The seed culture was used to inoculate glass jars containing 19 g solid substrate medium HiR2 (see Table 2) at a ratio of 950 μl seed per jar. The containers were incubated at 25° C. in a humidified atmosphere for 19 days.

TABLE 1
Recipe of MAT2 medium

| Ingredients | Amount | |
|---|---|---|
| malt extract | 15.0 | g |
| Tween 82 | 2.0 | ml |
| agar agar | 1.0 | g |
| demin. Water | 1000 | ml |

pH 7.0

TABLE 2
Recipe of HiR2 medium

| Ingredients | Amount | |
|---|---|---|
| brown rice | 240 | g |
| millet flakes | 240 | g |
| soy peptone | 3.0 | g |
| MgCl₂ × 6H₂O | 0.3 | g |
| demin. Water | 256 | ml |

No pH adjustment

Solid substrate cultures from around 750 g inoculated HiR2 medium were harvested by freezing at −20° C. For extraction the cultures were overlayed with methanol (100 ml), homogenized, and three times extracted with methanol (1500 ml each). The extract of 33.9 g dry weight was evaporated to dryness with double the amount of Celite.

The crude extract was fractionated by RP-MPLC according to Method 1. Fraction C (analyzed with Method 2) with retention time window from 25.5 to 33 min was evaporated to yield 1.3 g of pre-purified extract. Fraction C was further purified by RP-HPLC according to Method 5.

Fractions 30, 31 and 32 yielded 18.8 mg of “A4” as an amorphous solid in 89.3% purity (Method 6), Rt (Method 6)=15.28 min;

ESIMS (+): m/z=482 [M+Na]⁺, 470 [M+H]⁺, 452 [M−H₂O+H]⁺;

¹H-NMR (CD₃OD, 400 MHz) and HH—COSY, HMBC, HMQC (CD₃OD, 500 MHz):

| Position | ¹³C [ppm] | ¹H [ppm] | | J [Hz] |
|---|---|---|---|---|
| 1 | — | — | | |
| 2 | 178.6 | — | | |
| 3 | 54.3 | 4.79 | dd | 9.5; 7.0 |
| 4 | — | — | | |
| 5 | 172.5 | — | | |
| 6 | 38.4 | 2.63 | m | |
| 7 | 42.4 | 2.32 | m | 17.0 |
| | | 1.82 | brd | |
| 8 | 134.7 | — | | |
| 9 | 123.8 | 5.04 | brd | 9.0 |
| 10 | 34.8 | 2.70 | m | |
| 11 | 78.6 | 3.89 | s | |
| 12 | 138.1 | — | | |
| 13 | 127.6 | 5.34 | brd | 9.0 |
| 14 | 37.1 | 2.75 | m | |
| 15 | 82.0 | 4.75 | dd | 7.0, 4.5 |
| 16 | 34.8 | 2.34 | m | |
| 17 | 27.0 | 0.78 | m | |
| | | 1.02 | m | |
| 18 | 13.2 | 0.76 | t | 6.5 |
| 19 | 18.6 | 0.91 | | 7.0 |
| 20 | 39.5 | 3.09 | dd | 14.0; 7.0 |
| | | 2.86 | dd | 14.0; 9.5 |
| 21 | 138.0 | — | | |
| 22 | 127.6 | 7.17 | m | |
| 23 | 127.6 | 7.17 | m | |
| 24 | 129.8 | 7.24 | m | |
| 25 | 20.3 | 0.91 | | 6.5 |
| 26 | 18.6 | 1.58 | brs | |
| 27 | 12.1 | 0.79 | | 6.5 |
| 28 | 15.8 | 1.51 | brs | |
| 29 | 18.67 | 1.03 | d | 7.0 |

### Biological Activity of New Natural Products

The activity of the natural products, with respect to their TGR5 activating capacities, was analyzed as follows:

BHK cells, expressing the human TGR5, were seeded into 384-well plates and grown overnight. The next day, natural test compounds (and LCA as positive control) were incubated on the cells for 30 minutes. As TGR5 couples to Gi proteins, the increase of cellular cAMP production, as a measure for TGR5 activity, was analyzed by using a commercially available cAMP kit (CisBio international). The cAMP production was measured according to the kit manufacturer's protocol. Data were normalized by setting the cAMP production induced by LCA to 100% activity. EC50 values were determined by using the GraphPad PrismSoftware (Graph. Pad Software Inc.).

| | | TGR5 |
|---|---|---|
| | Structure | (EC50) |
| “A3” | 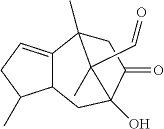 | 1 · 10⁻⁵ M |
| “A1” | 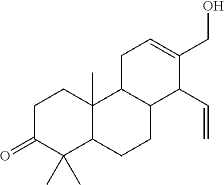 | 3 · 10⁻⁶ M |
| “A2” | 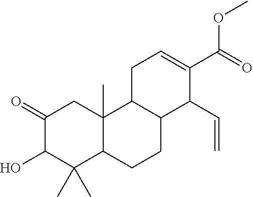 | 1 · 10⁻⁵ M |
| “A4” | 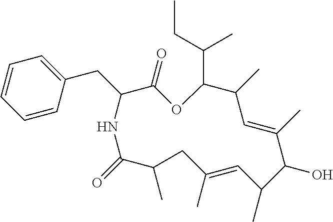 | 4 · 10⁻⁷ |

### EXAMPLE 5

### 1-Ethyl-4-b,8,8-trimethyl-7-oxo-tetradecahydro-phenanthrene-2-carboxylic acid dimethylamide (“A5”)

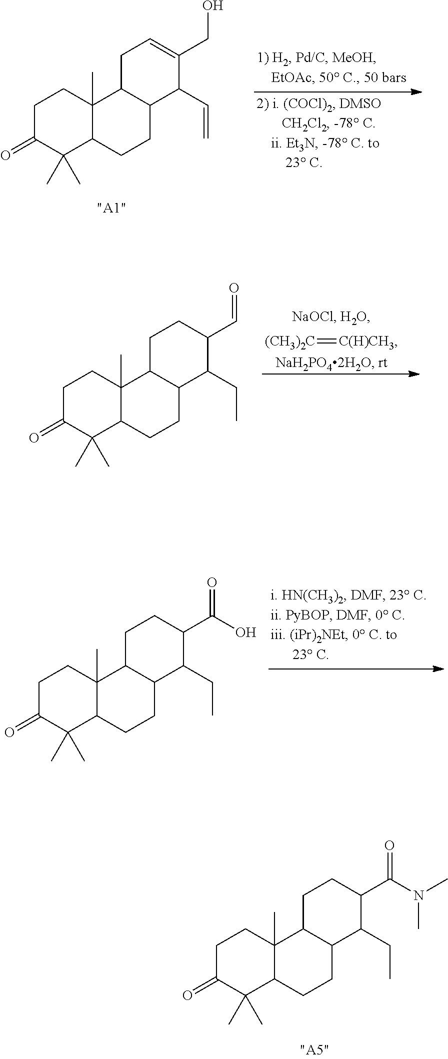

### EXAMPLE 6

### 7-Hydroxy-4-b,8,8-trimethyl-6-oxo-1-vinyl-tetradecahydro-phenanthrene-2-carboxylic acid (2-amino-ethyl)-amide (“A6”)

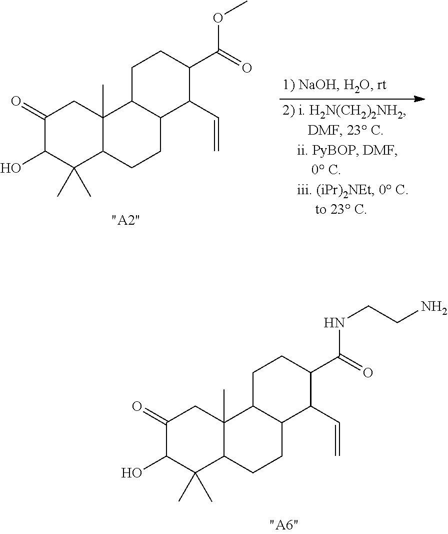

### EXAMPLE 7

### 1,4,9-Trimethyl-9-methylaminomethyl-2,4,5,6,8,8a-hexahydro-1H-4 methano-azulene-6,7-diol (“A7”)

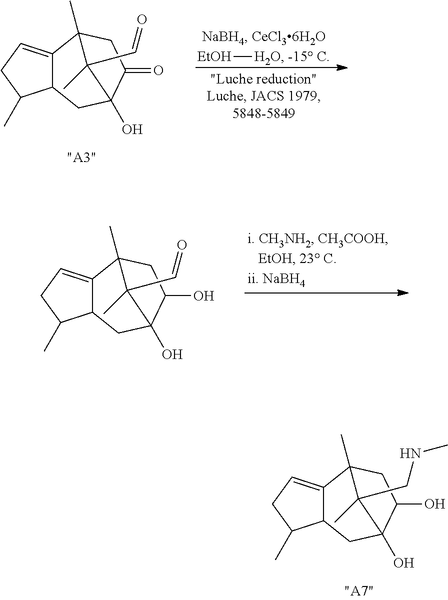

### EXAMPLE 8

### 1′-Amino-3-benzyl-15-sec-butyl-6,8,10,12,14-pentamethyl-1-oxa-4-azacyclopentadecane-2,5-dione (“A8”)

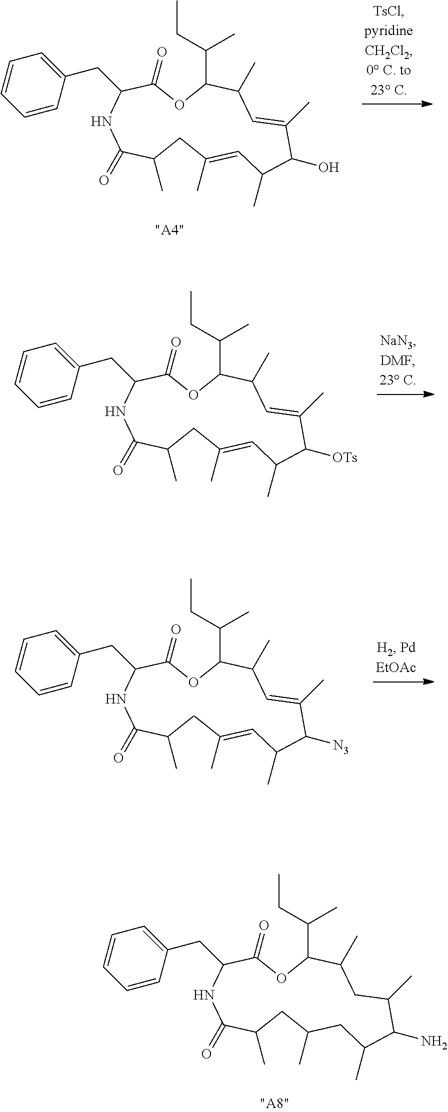

The following examples relate to pharmaceutical preparations:

### EXAMPLE A

### Injection Vials

A solution of 100 g of an active ingredient according to the invention and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient according to the invention with 100 g of soya lecithin and 1400 g of cocoa butter is melted, poured into moulds and allowed to cool. Each suppository contains 20 mg of active ingredient.

### EXAMPLE C

### Solution

A solution is prepared from 1 g of an active ingredient according to the invention, 9.38 g of NaH₂PO₄.2 H₂O, 28.48 g of Na₂HPO₄.12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient according to the invention are mixed with 99.5 g of Vaseline under aseptic conditions.

### EXAMPLE E

### Tablets

A mixture of 1 kg of active ingredient according to the invention, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

### Dragees

Tablets are pressed analogously to Example E and subsequently coated in a conventional manner with a coating of sucrose, potato starch, talc, tragacanth and dye.

### EXAMPLE G

### Capsules

2 kg of active ingredient according to the invention are introduced into hard gelatine capsules in a conventional manner in such a way that each capsule contains 20 mg of the active ingredient.

### EXAMPLE H

### Ampoules

A solution of 1 kg of an active ingredient according to the invention in 60 l of bidistilled water is sterile filtered, transferred into ampoules, lyophilised under sterile conditions and sealed under sterile conditions. Each ampoule contains 10 mg of active ingredient.

1. Compounds selected from the group
a) compounds of formula I 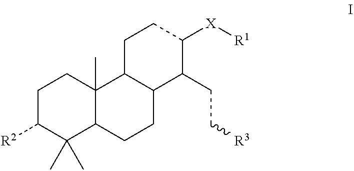
in which
R¹ denotes OH, OA, OAr, OC(O)A, OC(O)Ar, NH₂, NHA, NA₂, NHAr, NHC(O)A or NHC(O)Ar,
R² denotes O, OH, OA, OAr, OC(O)A, OC(O)Ar, NH₂, NHA, NA₂, NHAr, NHC(O)A or NHC(O)Ar,
R³ denotes H, A or Ar,
X denotes CH₂ or CO,
R⁴ denotes H or alkyl having 1-4 C atoms, [symbol]
denotes a single or double bond,
A denotes unbranched or branched alkyl having 1-10 C atoms, in which 1-7 H atoms may be replaced by F and/or Cl,
Ar denotes phenyl, naphthyl or biphenyl, each of which is unsubstituted or mono-, di- or trisubstituted by Hal, A, OR⁴, N(R⁴)₂, NO₂, CN, COOR⁴, CON(R⁴)₂, NR⁴COA, NR⁴SO₂A, COR⁴, SO₂N(R⁴)₂, S(O)ₙA, [C(R⁴)₂]ₘCOOR⁴ and/or O[C(R⁴)₂]ₘCOOR⁴,
m denotes 0, 1, 2, 3 or 4,
n denotes 0, 1 or 2,
Hal denotes F, Cl, Br or I;
b) compounds of formula II 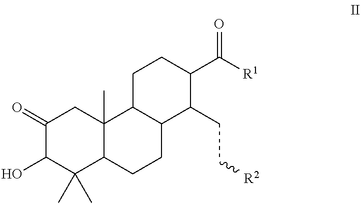
in which
R¹ denotes OH, OA, OAr, NH₂, NHA, NA₂, NHAr, NH(CH₂)ₚNH₂, NH(CH₂)ₚNHA, NH(CH₂)ₚNA₂,
R² denotes H, A or Ar,
R⁴ denotes H or alkyl having 1-4 C atoms, [symbol]
denotes a single or double bond,
A denotes unbranched or branched alkyl having 1-10 C atoms, in which 1-7 H atoms may be replaced by F and/or Cl,
Ar denotes phenyl, naphthyl or biphenyl, each of which is unsubstituted or mono-, di- or trisubstituted by Hal, A, OR⁴, N(R⁴)₂, NO₂, CN, COOR⁴, CON(R⁴)₂, NR⁴COA, NR⁴SO₂A, COR⁴, SO₂N(R⁴)₂, S(O)ₙA, [C(R⁴)₂]ₘCOOR⁴ and/or O[C(R⁴)₂]ₘCOOR⁴,
m denotes 0, 1, 2, 3 or 4,
n denotes 0, 1 or 2,
p denotes 1, 2, 3 or 4,
Hal denotes F, Cl, Br or I;
c) compounds of formula III 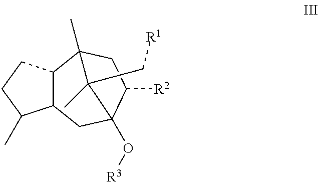
in which
R¹ denotes O, OH, OA, OAr, OC(O)A, OC(O)Ar, NH₂, NHA, NA₂, NHAr, NHC(O)A or NHC(O)Ar,
R² denotes O, OH, OA, OAr, OC(O)A, OC(O)Ar, NH₂, NHA, NA₂, NHAr, NHC(O)A or NHC(O)Ar,
R³ denotes H, A or Ar,
R⁴ denotes H or alkyl having 1-4 C atoms, [symbol]
denotes a single or double bond,
A denotes unbranched or branched alkyl having 1-10 C atoms, in which 1-7 H atoms may be replaced by F and/or Cl,
Ar denotes phenyl, naphthyl or biphenyl, each of which is unsubstituted or mono-, di- or trisubstituted by Hal, A, OR⁴, N(R⁴)₂, NO₂, CN, COOR⁴, CON(R⁴)₂, NR⁴COA, NR⁴SO₂A, COR⁴, SO₂N(R⁴)₂, S(O)ₙA, [C(R⁴)₂]ₘCOOR⁴ and/or O[C(R⁴)₂]ₘCOOR⁴,
m denotes 0, 1, 2, 3 or 4;
n denotes 0, 1 or 2,
Hal denotes F, Cl, Br or I;
d) compounds of formula IV 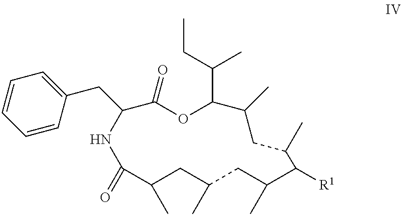
in which
R¹ denotes OH, OA, OAr, OC(O)A, OC(O)Ar, NH₂, NHA, NA₂, NHAr, NHC(O)A or NHC(O)Ar,
R⁴ denotes H or alkyl having 1-4 C atoms, [symbol]
denotes a single or double bond,
A denotes unbranched or branched alkyl having 1-10 C atoms, in which 1-7 H atoms may be replaced by F and/or Cl,
Ar denotes phenyl, naphthyl or biphenyl, each of which is unsubstituted or mono-, di- or trisubstituted by Hal, A, OR⁴, N(R⁴)₂, NO₂, CN, COOR⁴, CON(R⁴)₂, NR⁴COA, NR⁴SO₂A, COR⁴, SO₂N(R⁴)₂, S(O)ₙA, [C(R⁴)₂]ₘCOOR⁴ and/or O[C(R⁴)₂]ₘCOOR⁴,
m denotes 0, 1, 2, 3 or 4,
n denotes 0, 1 or 2,
Hal denotes F, Cl, Br or I;
and pharmaceutically usable salts and stereoisomers thereof, including mixtures thereof in all ratios.

2. Compounds according to claim 1 selected from the group
a) compounds of formula Ia 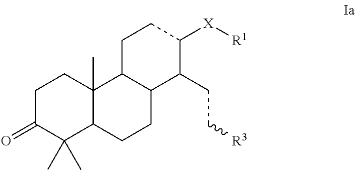
in which
R¹ denotes OH, OA, NH₂, NHA or NA₂,
R³ denotes H or A,
X denotes CH₂ or CO, [symbol]
denotes a single or double bond,
A denotes unbranched or branched alkyl having 1-10 C atoms, in which 1-7 H atoms may be replaced by F and/or Cl;
b) compounds of formula IIa 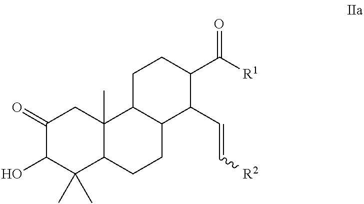
in which
R¹ denotes OH, OA, NH₂, NHA, NA₂, NH(CH₂)ₚNH₂, NH(CH₂)ₚNHA, NH(CH₂)ₚNA₂,
R² denotes H or A, [symbol]
denotes a single or double bond,
A denotes unbranched or branched alkyl having 1-10 C atoms, in which 1-7 H atoms may be replaced by F and/or Cl,
p denotes 1, 2, 3 or 4;
c) compounds of formula IIIa 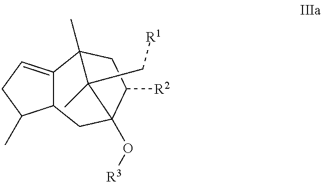
in which
R¹ denotes O, OH, OA, NH₂; NHA or NA₂,
R² denotes O, OH or OA,
R³ denotes H or A, [symbol]
denotes a single or double bond,
A denotes unbranched or branched alkyl having 1-10 C atoms, in which 1-7 H atoms may be replaced by F and/or Cl;
d) compounds of formula IV 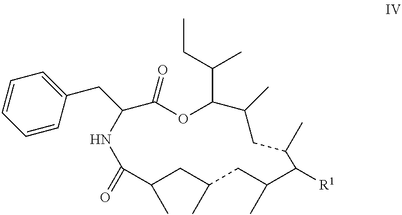
in which
R¹ denotes OH, OA, NH₂, NHA or NA₂, [symbol]
denotes a single or double bond,
A denotes unbranched or branched alkyl having 1-10 C atoms, in which 1-7 H atoms may be replaced by F and/or Cl;
and pharmaceutically usable salts and stereoisomers thereof, including mixtures thereof in all ratios.

3. Compounds according to claim 1 selected from the group

| no. | name and/or structure |
|---|---|
| “A1” | 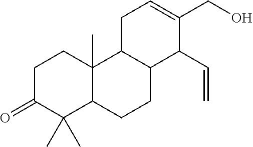 |
| “A2” | 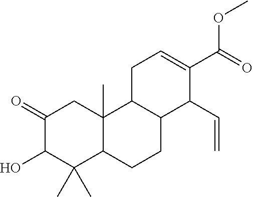 |
| “A3” | 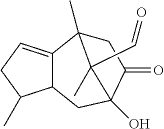 |
| “A4” | 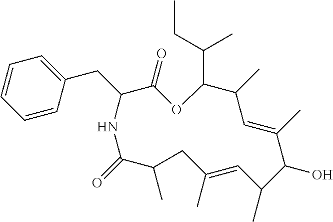 |
| “A5” | 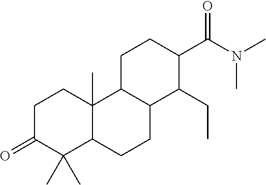 |
| “A6” | 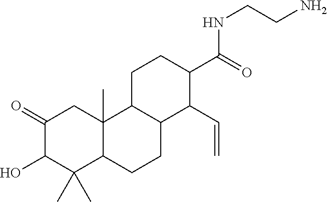 |
| “A7” | 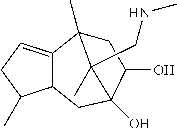 |
| “A8” | 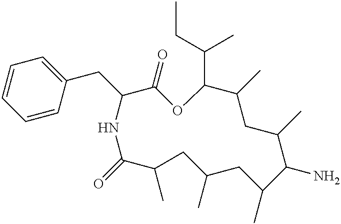 |

and pharmaceutically usable salts and stereoisomers thereof, including mixtures thereof in all ratios.

4. Process for the preparation of compounds of the formula I, II and IV of claim 1 and pharmaceutically usable salts and stereoisomers thereof,
characterised in that
a radical R¹ is converted into another radical R¹ by converting a hydroxyl or alkoxy group to an optionally substituted amino group
and/or
a base or acid of the formula I, II or IV is converted into one of its salts.

5. Process for the preparation of compounds of the formula III of claim 1 and pharmaceutically usable salts and stereoisomers thereof,
characterised in that
a carbonyl group is converted by reductive amination into an optionally substituted amino group
and/or
a base or acid of the formula III is converted into one of its salts.

6. Medicaments comprising at least one compound according to claim 1 and/or pharmaceutically usable salts and stereoisomers thereof, including mixtures thereof in all ratios, and optionally excipients and/or adjuvants.

7. Use of compounds according to claim 1 and pharmaceutically usable salts and stereoisomers thereof, including mixtures thereof in all ratios, for the preparation of a medicament for the treatment of a disease or condition, where the disease or condition is insulin-dependent diabetes mellitus, non-insulin-dependent diabetes mellitus, obesity, neuropathy and/or nephropathy.

8. Medicaments comprising at least one compound according to claim 1 and/or pharmaceutically usable salts and stereoisomers thereof, including mixtures thereof in all ratios, and at least one further medicament active ingredient.

9. Set (kit) consisting of separate packs of
(a) an effective amount of a compound according to claim 1 and/or pharmaceutically usable salts and stereoisomers thereof, including mixtures thereof in all ratios,
and
(b) an effective amount of a further medicament active ingredient.
